(19) United States
(12) Patent Application Publication
(10) Pub. No.: US 2020/0046927 A1
(43) Pub. Date: Feb. 13, 2020

(54) BREATHING ASSISTANCE APPARATUS AND RELATED METHODS

(71) Applicant: Joseph Batrice, Lubbock, TX (US)

(72) Inventor: Joseph Batrice, Lubbock, TX (US)

(21) Appl. No.: 16/057,777

(22) Filed: Aug. 7, 2018

(51) Int. Cl. A61M 16/06; A61M 16/00; A61M 16/20

(52) U.S. Cl. CPC A61M 16/0666; A61M 16/209; A61M 16/0057

(57) ABSTRACT

Breathing disorders such as snoring and sleep apnea are common disorders disrupting an individual's normal breathing. Improper or obstructed breathing can affect a person's health and wellbeing. This application describes a breathing assistance apparatus and related methods for opening a person's nasal passage to aide in breathing and treat common breathing disorders.

### CROSS-REFERENCE TO RELATED APPLICATIONS

None.

### STATEMENT OF FEDERALLY SPONSORED RESEARCH OR DEVELOPMENT

None.

### REFERENCE TO A SEQUENCE LISTING, A TABLE, OR A COMPUTER LISTING COMPACT DISC APPENDIX

None.

### BACKGROUND OF THE INVENTION

This invention relates to a device and method of breathing assistance.

Snoring is the vibration of respiratory structures and the resulting sound due to obstructed air movement during breathing while sleeping. In some cases, the sound may be soft, but in most cases, it can be loud and unpleasant. Snoring during sleep may be a sign, or first alarm, of very serious breathing disorders.

Obstructive respiratory disorders entail apnea (respiratory arrest) causing the sleeping person to wake up. Frequent apnea prevents the sleeping sufferer from entering recuperative deep sleep. As a result, sufferers incurring apnea during their sleep are sleep-deprived during the day: social problems may arise at work and in the worst case fatal accidents may be incurred.

Devices for carrying out the therapy of Continuous Positive Airway Pressure (CPAP) are known in the state of the art. However, these machines can be bulky and cumbersome to use. In addition, a sleep study must often be conducted before a user may obtain a CPAP device.

In CPAP therapy, a constant, excess pressure is fed to a patient through a nasal mask. When the excess pressure is properly selected, it keeps the upper airway fully open overnight and as a result obstructive respiratory disorders do not arise. In part the required pressure depends on the sufferer's sleep stage and his/her body position.

Breathing assistance devices that do not use positive airway pressure are known in the art. Some are placed in the user's mouth (See U.S. Pat. No. 5,277,202 an “Easy fit anti-snoring device” and U.S. Pat. No. 3,132,647 an “Anti-snoring device”); some are even fitted to the user's head (See U.S. Pat. No. 7,331,349 a “Method and device for the prevention of snoring and sleep apnea”); and some are fitted in the user's nostrils (See U.S. Pat. No. 2,672,138 a “Device to promote nasal breathing and prevent snoring”). However, all these devices have their limitations in that they become loose during the night or even fall completely out of place, rendering them useless. Therefore, a need exists for a fitted breathing apparatus that stays in place and will assist in the user's breathing throughout the night and motion of the user.

In this respect, the breathing assistance system, specifically the apparatus and methods, according to the present invention substantially departs from the conventional concepts and designs of the prior art, and in doing so provides an apparatus primarily developed for the purpose of allowing a user to easily combat a snoring problem or other sleep or breathing disorder.

Therefore, it can be appreciated that there exists a continuing need for a new and easy to use breathing assistance device for use in combatting snoring and other breathing disorders. In this regard, the present invention substantially fulfills this need.

### SUMMARY OF THE INVENTION

In one embodiment, the claimed invention is a self-stabilizing breathing assistance apparatus comprising a pump apparatus, a plurality of tubes for insertion through the nostrils into the nasal passageway and a release valve capable of releasing the gas or liquid within the apparatus or plugging the gas or liquid within the apparatus. A gas such as air or oxygen may be used in particular embodiments. A liquid such as water may be used in particular embodiments.

In one particular embodiment, the pump apparatus may comprise a pump, a valve, and an inflation chamber. The pump is capable pumping a gas or a liquid. In a preferred embodiment, the valve is a check valve. In other embodiments, the valve may be a clack valve, non-return valve, reflux valve, or one-way valve. The valve is capable of receiving a pump that unilaterally pumps a gas or a liquid through the valve into an inflation chamber. In inflation chamber may be comprised of a rigid and supportive material such as hard plastic or metal.

There is at least one inner tube and at least one outer tube. In a preferred embodiment, the inner tube is semi rigid and composed of a bendable plastic so as to bend through the user's nostril and into the nasal passageway. In a preferred embodiment, the outer tube is flexible and inflatable. The outer tube may be comprised of an inflatable material such as plastic or rubber.

The outer tube is sealed at the distal end and attached to the inflation chamber at the proximal end, thereby allowing sealed inflation by the pump apparatus. The device is inserted through the user's nostril and into the user's nasal passageway. Once inserted in its deflated state, the device may then be inflated to affix itself inside the user's nostril and nasal passageway. When inflated, the outer tube is rigid and pressed against the user's nasal passageway thereby anchoring the apparatus and allowing a fixed breathing passageway for the user through the inner tube.

Use of the claimed device includes a method of opening the nasal passageway for increased and consistent airflow. The method comprises insertion of the un-inflated device through the user's nostril or nostrils and into the user's nasal passageway. The device may be between 1 and 4 inches in length depending on the size of the user's nose and the user's preferred depth of insertion.

Once the un-inflated device is inserted, the pump may be attached to the valve. The pump then pumps a gas or liquid through the valve and into the inflation chamber thereby filling the inflation chamber and the attached outer tube or tubes. As the outer tube or tubes fill, they inflate to press against the walls of the user's nasal passageway and the user's nostril. In one embodiment, the pump may be operated until the outer tube or tubes touch the inner walls of the user's nostril and nasal passageway. In another embodiment, the outer tube or tubes may be inflated further to physical expand the user's nostril and nasal passageway.

In a preferred embodiment, the outer tube or tubes are inflated an amount sufficient to hold the device in the user's nose. At which point, the pump may be removed from the valve. The user may then freely breathe through the inner tube or tubes of the device.

In a preferred embodiment, the user may remove the device by un-inflating the outer tube or tubes and pulling the device out of the user's nasal passageway and nostril. In one embodiment, un-inflation may be accomplished by removing a plug on the inflation chamber. In another embodiment, un-inflation may be accomplished by activating a release on the valve.

### BRIEF DESCRIPTION OF THE DRAWINGS

These and other features, aspects, and advantages of the present invention will become better understood with regard to the following descriptions, appended claims, and accompanying drawings where:

FIG. 1A shows the inflation view of the apparatus.

FIG. 1B shows a direct view of the breathing tubes.

FIG. 2 shows the insertion of the apparatus into a user's nostrils.

FIG. 3 shows a detailed view of insertion of the apparatus into a user's nasal passageway.

### DETAILED DESCRIPTION OF THE INVENTION

In the Summary of the Invention above and in the Detailed Description of the Invention, and the claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components.

Where reference if made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm.

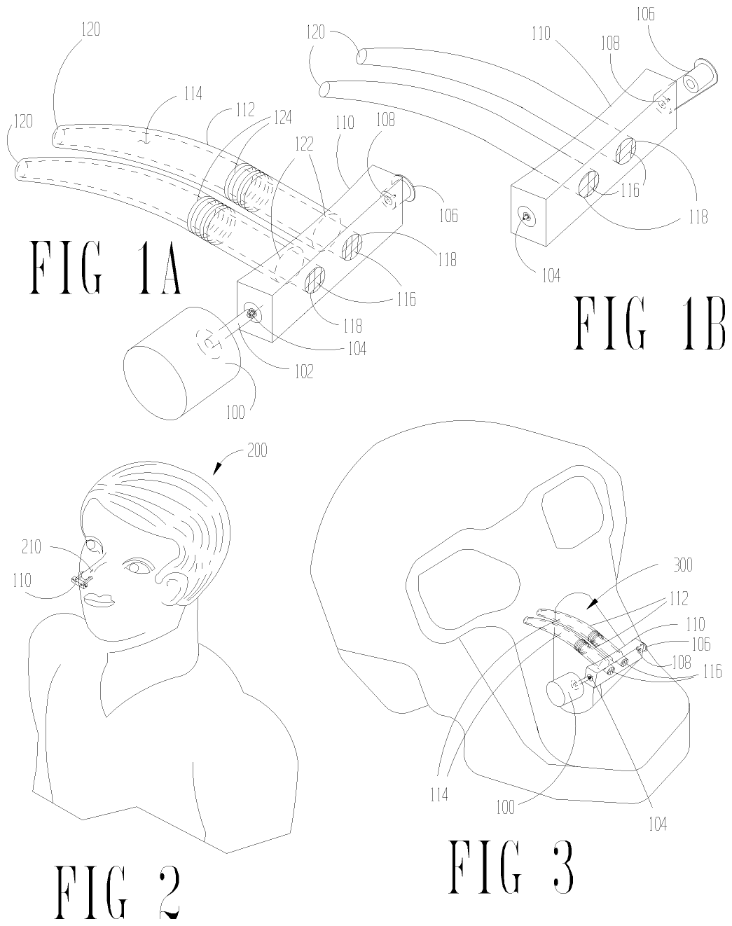

As shown in FIG. 1A-1B, one embodiment of the claimed apparatus and methods comprises a pump 100 with a check valve attachment 102. In certain embodiments the pump 100 may be a mechanical air pump or electrical air pump to pump air into the device and the inflation chamber 110. In certain embodiments, the pump 100 may be a user-controlled or autonomous. The pump 100 is operated to pump gas or liquid through the check valve attachment 102 into the check valve 104. Once the gas or liquid is pumped through the check valve 104 by the pump 100, the inflation chamber 110 of the device is filled.

As shown in FIG. 1A-1B, the inflation chamber 110 is built around a plurality of breathing tubes 114. In a preferred embodiment, the inflation chamber 110 is near the proximal end 118 of the breathing tubes 114. The number of breathing tubes 114 corresponds to the number of nostrils or nasal cavity openings a user requires assistance breathing through.

In a preferred embodiment, the breathing tubes 114 are semi-rigid. The breathing tubes 114 are further structurally supported by the inflation chamber 110. The proximal end 118 of the breathing tubes 114 are divided from the distal end 120 of the breathing tubes 114 by the inflation chamber 110. The internal tubes 114 are hollow such that air can easily travel through them as the user breathes. Said air may be oxygen, carbon dioxide, or a mixture as the user breathes and inhales and exhales. The breathing tubes 114 protrude through the inflation chamber 110, exposing their proximal ends 118 and hollow openings 116 to the user's environment. In a preferred embodiment, air travels through the breathing tubes 114 from the proximal end 118 to the distal end 120 and into the user's nasal cavity as the user inhales. As the user exhales, air travels from the user's nasal cavity through the breathing tubes 114 from the distal end 120 to the proximal end 118 and out into the user's environment through the exposed hollow openings 116. As depicted in FIG. 1A-1B, the hollow openings 116 may be covered by screens to allow unrestricted airflow, but to keep debris or particles from entering the breathing tubes.

In a preferred embodiment, the distal end 120 of the breathing tubes tapers such that it is narrower than the proximal end 118. This may allow for easier insertion through the user's nostril and into the nasal cavity 300 as many user's nasal cavity may narrow as it deepens. Also in a preferred embodiment, the breathing tubes 114 are curved from their proximal end 118 to their distal end 120, allowing easier insertion through the user's nostrils and into the nasal cavity 300.

As shown in FIG. 1A-1B, in a preferred embodiment, the breathing tubes 114 are surrounded by inflation tubes 112 on the breathing tubes distal end 120. The inflation tubes 112 may be comprised of any material that is flexible and does not allow gas or liquid to permeate it. Specifically, FIG. 1A depicts one embodiment of the breathing tubes in which they are comprised of support structures 124. In one embodiment, these support structures 124 may be ridges to keep the breathing tubes from collapsing.

In one embodiment, the inflation tubes 112 are sealed at their distal end to the breathing tubes 114 such that gas or liquid remains once it is pumped into the inflation tubes 112. In a preferred embodiment, the inflation tubes 112 are sealed on their outer edges to the outer edge of the inflation chamber 122. This allows the exposed chamber created within the inflation tubes 112 to be exposed to the inflation chamber 110.

In a preferred embodiment, as the inflation chamber 110 is filled with gas or liquid, the cavity between the breathing tubes 114 and the inflation tubes 112 fills with said gas or liquid. In a preferred embodiment, gas or liquid is pumped by a pump 100 unidirectionally through the check valve 104 into the inflation chamber 110 and into the inflation tubes 112. In one embodiment, the inflation tubes 112 are comprised of an expanding material that expands as gas or liquid is pumped into the inflation tubes 112. When gas or liquid is pumped into the inflation chamber 110 and inflation tubes 112, it is trapped from deflating by the check valve.

As shown in FIG. 1A-1B, in one embodiment, the inflation chamber may comprise of a release opening 108. In a preferred embodiment, the release hole 108 may be capped or plugged by a release plug 106 such to keep gas or liquid contained within the inflation chamber 110 and inflation tubes 112.

In one embodiment, the release plug 106 may be removed or inserted into the release hole 108 by mechanical or electrical means. In one embodiment the release plug 106 may be removed or inserted into the release hole 108 by the user or may be controlled autonomously.

In another embodiment, gas or liquid may be released from the inflation chamber 110 and inflation tubes 112 by the check valve 104. In such an embodiment, the check valve would comprise of a release mechanism. In one embodiment, the check valve release mechanism may be operated mechanically or electrically. In one embodiment, the check valve release mechanism may be operated by the user or autonomously.

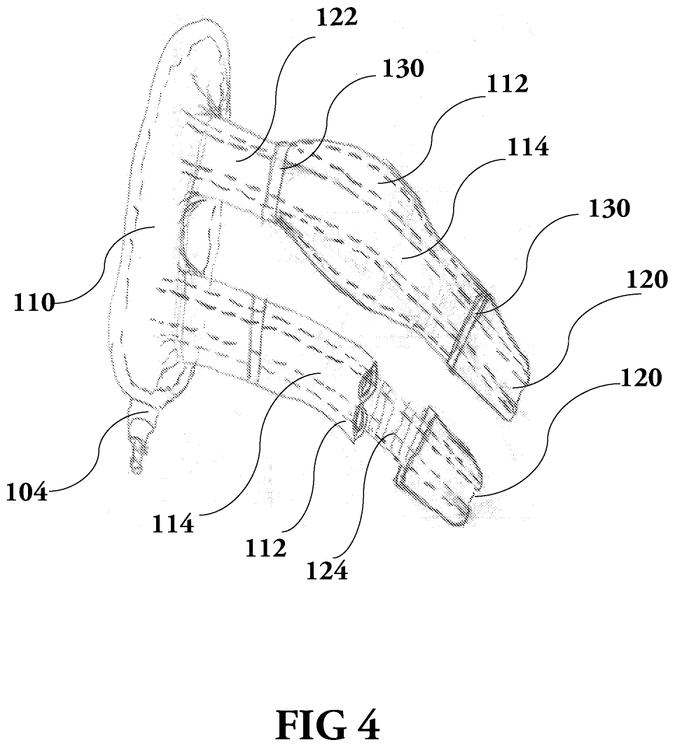

As depicted in FIG. 2, the distal ends of the breathing tubes 120 are inserted into the nostrils 210 of a user 200. In one embodiment, a device with breathing tubes 114 only may be inserted. In preferred embodiment, the breathing tubes 114 and inflation tubes 112 are inserted in their deflated form into the user's nostrils 210. In one embodiment, the claimed invention may have only one breathing tube 114 and one inflation tube 112 if the user only requires breathing assistance through one nostril. In another embodiment, the claimed invention has two breathing tubes 114 and two inflation tubes 112, one for each of the user's nostrils.

As depicted in FIG. 3, in one embodiment, the distal end of the deflated inflation tubes 112 and breathing tubes 114 are inserted through the user's nostrils and into the user's nasal passage 300. In one embodiment, the length of the breathing tubes 114 and inflation tubes 112 may vary based on the size of the user's nose, nostrils, and nasal passage.

In a preferred embodiment, once the breathing tubes 114 and inflation tubes 112 have been inserted into the nasal passageway 300, the pump 100 is then activated to pump gas or liquid unilaterally through the check valve 104 and into the inflation chamber 110. As the inflation chamber 110 fills with gas or liquid, the inflation tubes 112 inflate with said gas or liquid. As the inflation tubes 112 inflate, they expand and press against the lining of the nasal passageway 300.

In one embodiment, as the inflation tubes 112 press against the nasal passageway 300, the nasal passageway 300 is further opened or expanded. In a preferred embodiment, once the inflation tubes 112 are inflated enough to secure themselves to the nasal passageway 300, then the pump 100 may be removed from the check valve 104. In another embodiment, the pump 100 is permanently affixed to the check valve 104.

In a preferred embodiment, as the inflation tubes 112 expand against the nasal passageway 300, they secure the entire apparatus to the nose. Once secured, the user can breathe through the breathing tubes 114 without the breathing tubes 114 shifting or falling out of the user's nasal passageway 300 or nostrils 210.

In one embodiment, the breathing tubes 114 and inflation tubes 112 are removed from the user's nostrils 210 and nasal passageway by deflating the inflation tubes 112 and freeing the device. In one embodiment, said deflation may occur by removing a release plug 106 from a release hole 108 on the inflation chamber 110. In another embodiment, said deflation may occur by engaging a release mechanism on the check valve 104.

Once deflated, the inflation tubes 112, breathing tubes 114, and entire apparatus may be removed from the user's nasal cavity 300 and nostrils 210.

1. A self-stabilizing breathing assistance apparatus comprising:
a pump apparatus;
a plurality of tubes for insertion through the nostrils into the nasal passageway to anchor the breathing assistance apparatus to only the middle section of the nasal passageway; and
a release valve capable of releasing a gas or liquid within said breathing assistance apparatus or plugging the gas or liquid within the breathing assistance apparatus.

2. The self-stabilizing breathing assistance apparatus of claim 1, wherein said pump apparatus comprises:
a pump;
a valve; and
an inflation chamber.

3. The self-stabilizing breathing assistance apparatus of claim 2, wherein the pump is capable of pumping gas or liquid.

4. The self-stabilizing breathing assistance apparatus of claim 2, wherein the valve is a check valve capable of receiving said pump that unilaterally pumps gas or liquid through the valve into an inflation chamber comprised of a supportive material.

5. The self-stabilizing breathing assistance apparatus of claim 1, further comprising the tubes of claim 1 wherein there is at least one inner tube and one outer tube.

6. The self-stabilizing breathing assistance apparatus of claim 5, wherein the inner tube is semi rigid and comprised of support structures.

7. The self-stabilizing breathing assistance apparatus of claim 5, wherein the outer tube is flexible and inflatable consistently around the inner tube to form a symmetrical cylindrical shape when inflated.

8. The self-stabilizing breathing assistance apparatus of claim 5, wherein the outer tube is sealed at the outer tube's distal end and attached to an inflation chamber at the outer tube's proximal end, thereby allowing sealed inflation by the pump apparatus.

9. The self-stabilizing breathing assistance apparatus of claim 8, wherein when inflated, the outer tube becomes firm and is pressed against only the middle of the user's nasal passageway thereby anchoring the breathing assistance apparatus and allowing a fixed breathing passageway for the user through the inner tube.

10. A method of opening the nasal passageway for increased and consistent airflow to prevent snoring comprising:
inserting a plurality of tubes, said plurality of tubes comprising at least one semi rigid and supported inner tube and one flexible and inflatable outer tube through two nostrils into the nasal passageway;
using a pump apparatus capable of pumping gas or liquid to inflate at least one outer tube of the plurality of tubes with a gas or liquid to temporarily affix the tubes within the user's nose by pressing at least one outer tube against only the middle of the user's nasal passageway, wherein said pump apparatus comprises: a pump, an inflation chamber, and a check valve capable of receiving said pump that unilaterally pumps gas or liquid through the valve into said inflation chamber comprised of a rigid supportive material;
engaging a release valve to release the gas or liquid within the outer tube; and
removing the plurality of tubes from the nasal passageway and nostrils.

11. (canceled)

12. (canceled)

13. (canceled)

14. (canceled)

15. (canceled)

16. (canceled)

17. The method of claim 10, wherein the outer tube is sealed at the outer tube's distal end and attached to the inflation chamber at the outer tube's proximal end, thereby allowing sealed inflation by the pump apparatus.

18. The method of claim 10, wherein when inflated, the outer tube is rigid and pressed against only the middle of the user's nasal passageway thereby anchoring the breathing assistance apparatus and allowing a fixed breathing passageway for the user through the inner tube.
